(19) United States
(12) Patent Application Publication
(10) Pub. No.: US 2026/0314973 A1
(43) Pub. Date: Oct. 15, 2026

(54) LEVATOR ANI DEVICE

(71) Applicants: Catapult Products, LLC, Salt Lake City, UT (US); University of Utah Research Foundation, Salt Lake City, UT (US)

(72) Inventors: Noah M. Boekweg, Salt Lake City, UT (US); Nicholas O. Duval, Murray, UT (US); Dalton M. Daley, Salt Lake City, UT (US); Julia C. Denton, Salt Lake City, UT (US); Sadie C. Austin, Middleton, WI (US); Sonia G. Para, Salt Lake City, UT (US); Robert W. Hitchcock, Salt Lake City, UT (US)

(21) Appl. No.: 19/646,366

(22) Filed: Apr. 13, 2026

Related U.S. Application Data
(60) Provisional application No. 63/787,476, filed on Apr. 11, 2025.

(51) Int. Cl. A61B 5/22; A61B 1/303; A61B 5/00; A61B 90/00

(52) U.S. Cl. CPC A61B 5/227; A61B 1/303; A61B 5/4538; A61B 90/06; A61B 90/08; A61B 2090/064; A61B 2090/0807

(57) ABSTRACT

A device to quantify the strength of the pelvic floor muscles in a human. A speculum is associated with a handle to facilitate hand-held use of the device. First and second bills of the speculum may be interconnected by way of a guide mechanism to hold length axes of respective bills in a parallel configuration during transverse bill deflection under load. One workable guide mechanism includes a plurality of multi-bar mechanisms. Desirably, bills are restrained by the guide mechanism to resist relative displacement with respect to each other. A transducer is provided to convert an applied load on the speculum to a quantifiable output visible to an operator of the device.

### RELATED APPLICATIONS

This application claims the benefit under 35 U.S.C. 119(e) of the filing date of Provisional Application Ser. No. 63/787,476, filed Apr. 11, 2025, for “LEVATOR ANI DEVICE”.

### FIELD OF THE INVENTION

This invention relates to medical devices configured to measure force generated by pelvic muscles.

### STATE OF THE ART

Pelvic floor disorders are prevalent conditions that affect a significant portion of the female population and are commonly associated with weakening of the pelvic floor muscles. Such disorders include, but are not limited to, urinary incontinence, pelvic organ prolapse, and chronic pelvic pain. Early identification of pelvic floor muscle weakness can facilitate preventative treatment and improve patient outcomes; however, objective and accessible diagnostic tools remain limited.

Current clinical assessment of pelvic floor muscle strength relies on digital palpation performed by a specialized clinician. During this evaluation, a practitioner inserts one or more fingers into the vagina and subjectively estimates muscle strength based on perceived displacement and pressure during voluntary contraction. This approach is inherently subjective and may vary between providers, resulting in inconsistent measurements and reduced reliability.

Alternative diagnostic instruments exist, including pressure-based perineometers and electronic dynamometers. These devices may require electronic components, calibration, specialized training, or significant cost, which can limit adoption in routine clinical settings. Additionally, some pressure-based devices may measure forces generated by surrounding musculature or intra-abdominal pressure rather than isolating the pelvic floor muscles.

Advances in the art of tools for measuring or quantifying physical phenomena were discovered in a patentability search. Patent-related literature discovered in the search include European patent application No. 15382501.3, titled “Apparatus for measuring pelvic floor muscle strength”. United States issued U.S. Pat. No. 6,468,232, titled Method and apparatus for measuring properties of the pelvic floor muscles” was located. United States patent applications Nos. US2019/0104978 titled “Force measuring speculum” and US2014/0276232 titled “Skin tensionometer” were also discovered in the search. The entire disclosures of the aforementioned documents are hereby incorporated into this disclosure by reference.

### BRIEF SUMMARY OF THE INVENTION

This invention may be embodied to provide a Levator Ani Device (LeAD), which is a mechanically actuated diagnostic instrument designed to quantify the strength of the pelvic floor muscles in a human. The device is intended to evaluate the levator ani muscles, the largest muscle group in the pelvic floor, to aid in the diagnosis, screening, and monitoring of pelvic floor disorders. Desirably, a LeAD provides an objective, quantitative measurement of pelvic floor muscle strength, enabling standardized assessment.

Preferred embodiments of a LeAD are designed to be similar to a vaginal speculum to be seamlessly integrated into existing workflows. Such devices may be used by clinicians during routine gynecological examinations, primary care visits, urogynecologic evaluations, or pelvic therapy sessions. A working end of a device may be inserted into the vagina and the patient instructed to perform a voluntary contraction of the pelvic floor muscles (eg., a “Kegel”). Contraction of the muscles compresses an exemplary device's parallel bills, causing displacement of a mechanical linkage. In certain cases, this displacement may be transmitted to a calibrated spring mechanism that converts the applied force into a measurable output, which is displayed on a dial readout. The measurement corresponds to the maximum voluntary contraction force and may be used to assess muscle strength and monitor changes over time.

An exemplary embodiment constructed according to certain principles of the invention includes a housing configured to provide a handle for a speculum. A workable speculum includes a first bill and a second bill configured for insertion into a vagina. A first bill may be affixed as a cantilevered beam extension from the housing. In that case, a second bill may be coupled to the first bill and constrained by a guide mechanism to permit reciprocating transverse displacement there-between. Preferably, the guide mechanism is configured to urge a length axis of the second bill to remain in parallel with a corresponding length axis of the first bill. Further, the guide mechanism is desirably configured to resist relative axial displacement between the first bill and the second bill in a bill length direction during actuation of the speculum.

Embodiments generally include a transducer associated with the speculum to convert a transverse displacement between the first and second bills to a visibly perceptible signal for observation by an operator of the apparatus. A workable transducer includes a pointer arm coupled at a first end to a first arm pivot point referenced to ground. Here “ground” is intended to be interpreted in its broad application to conventional characterization of linkage mechanisms. In this case, ground may encompass the housing, an element of the cantilever anchor of the first bill, or directly on cantilevered structure of the first bill. The pointer arm of a workable transducer can be rotatable about the first arm pivot point responsive to relative transverse displacement between the first and second bills during actuation of the speculum.

A pointer arm may include an extended first arm length disposed between the first arm pivot point and an actuator end. A base end of the pointer arm may be configured to provide a drive link including a drive link length disposed between the first arm pivot point and a centerline of a driven axle. In that case, amplified deflection of the actuator end, corresponding to relative displacement between the first and second bills, is a function including ratio of first arm length to drive link length.

An embodiment desirably includes a spring affixed at one spring end to the pointer arm at an intermediate location between the first arm pivot point and the actuator end, and affixed at an opposite spring end to structure corresponding to ground. A workable spring is a tension spring. In certain cases, the spring may be disposed inside a handle portion of the housing.

A currently preferred transducer includes a scale disposed on a proximal end of the housing to visually indicate a quantified number corresponding to a force applied for actuation of the speculum. An exemplary scale includes an arcuate rail and a reading indicator carried for reciprocation on the rail. The actuator end of the pointer arm can be operably associated with the reading indicator to displace the indicator along the rail responsive to actuation of the speculum.

A preferred reading indicator is configured in harmony with the rail and pointer arm to temporarily maintain a displaced position of the reading indicator subsequent to actuation of the speculum by an applied load. One such reading indicator is configured in cooperation with the rail to permit an operator of the apparatus to return the reading indicator to an initial relative zero position subsequent to removal of the load.

One arrangement for a speculum includes a driven axle that is rotatably affixed to a middle beam disposed generally in parallel with and between a first speculum bill and a second speculum bill. A preferred guide mechanism to control bill displacement includes a first multi-bar mechanism arranged to couple the first bill to the middle beam such that length axes of the first bill and middle beam remain in parallel during actuation of the speculum. Also, a second multi-bar mechanism is arranged to couple the second bill to the middle beam such that length axes of the second bill and middle beam remain in parallel during actuation of the speculum. Preferably, the first and second multi-bar mechanisms are structured in harmony to maintain the middle beam substantially at a midpoint between the first and second bills. In any case, the guide mechanism is configured such that the driven axle is operably displaced responsive to a transverse displacement between the first and second bills. Therefore, displacement of the driven axle may be operably coupled to a transducer.

One exemplary embodiment according to certain principles of the invention includes a housing configured to provide a handle for handheld use of a speculum. A preferred speculum includes a first beam, a second beam, and a third beam. Relative displacement between the first, second, and third beams is controlled by a guide mechanism. A currently preferred guide mechanism includes three multi-bar mechanisms.

The first beam is generally affixed at a proximal end as a cantilevered extension from the housing. The second beam is coupled to the first beam by way of a plurality of primary link elements to form a first multi-bar mechanism. The third beam is disposed between the first and second beams and coupled to the first beam by way of the plurality of primary link elements to form a second multi-bar mechanism. Further, a transducer is associated with the speculum to convert a transverse displacement between the first and second beams to a visibly perceptible signal for observation by an operator of the apparatus.

This exemplary embodiment includes a plurality of secondary link elements coupled for rotation at respective secondary link ends to the second and third beams to form a third multi-bar mechanism. It is preferred for the first multi-bar mechanism to be constructed and arranged to maintain the first and second beams in a parallel and spaced-apart relation. Also, the second multi-bar mechanism is desirably constructed and arranged to maintain the first and third beams in parallel. Further, the third multi-bar mechanism is desirably constructed and arranged to maintain the second and third beams in parallel.

In an exemplary arrangement, primary link first ends are rotatably coupled to the first beam. Primary link middles are rotatably coupled to the third beam. Also, primary link second ends are slidably coupled to the second beam to permit primary link second ends to slide with respect to the second beam and in the length direction of the second beam. Link elements variously disposed between first, second, and third beams inter-cooperate to resist axial displacement of the first beam with respect to the second beam in a length direction of the first beam. Link elements variously disposed between first, second, and third beams inter-cooperate to cause axial displacement of the third beam with respect to the first and second beams and in a length direction of the third beam. In the currently most preferred arrangement, axial displacement of the third beam is directed proximally responsive to application of a load to compress the speculum.

Preferably, the transducer is carried by the housing and disposed for actuation by the third beam. A workable transducer includes a pointer arm coupled at a first end to a first arm pivot axle, which defines a pointer rotation axis that is stationary with respect to the housing. The pointer arm may be operably disposed for rotation about the pointer rotation axis responsive to relative transverse displacement between the first and second bills during actuation of the speculum. A spring may be configured and arranged to bias the first and second beams apart. That same spring may be coupled to the pointer arm to permit quantifiable displacement of an actuator end of the pointer arm responsive to a load applied to the speculum.

### BRIEF DESCRIPTION OF THE DRAWINGS

In the drawings, which illustrate what are currently considered to be the best modes for carrying out the invention:

FIG. 1 is a front view in perspective of an exemplary embodiment constructed according to certain principles of the invention;

FIG. 2 is a left side view in elevation thereof;

FIG. 3 is a right side view in elevation thereof;

FIG. 4 is a top view thereof;

FIG. 5 is a bottom view thereof;

FIG. 6 is a rear view thereof;

FIG. 7 is a front view thereof;

FIG. 8 is a close-up view of a distal speculum portion thereof;

FIG. 9 is the cross-section view indicated at section 9-9 in FIG. 8, and looking in the direction of the arrows;

FIG. 10 is the cross-section view indicated at section 10-10 in FIG. 8, and looking in the direction of the arrows;

FIG. 11 is the cross-section view indicated at section 11-11 in FIG. 8, and looking in the direction of the arrows, but with the pointer disposed at a first alternative position;

FIG. 12 is the cross-section view indicated at section 12-12 in FIG. 8, and looking in the direction of the arrows, but with the pointer disposed at a first alternative position;

FIG. 13 is the cross-section view indicated at section 13-13 in FIG. 8, and looking in the direction of the arrows, but with the pointer disposed at a second alternative position;

FIG. 14 is the cross-section view indicated at section 14-14 in FIG. 8, and looking in the direction of the arrows, but with the pointer disposed at a second alternative position;

FIG. 15 is an exploded front view in perspective of a speculum portion of the embodiment in FIG. 1;

FIG. 16 is the embodiment in FIG. 15, now partially in section;

FIG. 17 is a close-up side view of a proximal end of the housing with the side cover removed to permit observation of the pointer mechanism;

FIG. 18 is the embodiment in FIG. 17, with the pointer arm displaced responsive to a load applied to the speculum;

FIG. 19 is the embodiment in FIG. 18, with the pointer arm now displaced responsive to the load being relaxed a bit; and

FIG. 20 is the embodiment in FIG. 19, with the pointer arm now displaced to an initial position reflecting an unloaded condition.

### DETAILED DESCRIPTION OF THE ILLUSTRATED EMBODIMENTS

Reference will now be made to the drawings in which the various elements of the illustrated embodiments will be given numerical designations and in which the invention will be discussed so as to enable one skilled in the art to make and use the invention. It is to be understood that the following description is only exemplary of certain principles of the present invention, and should not be viewed as narrowing the claims which follow.

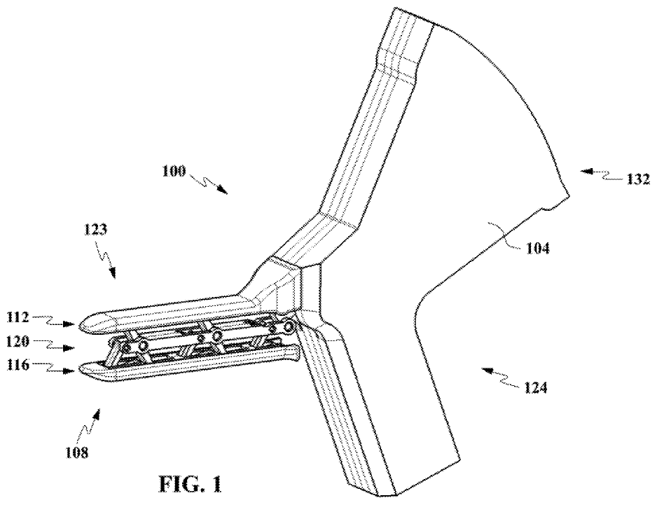

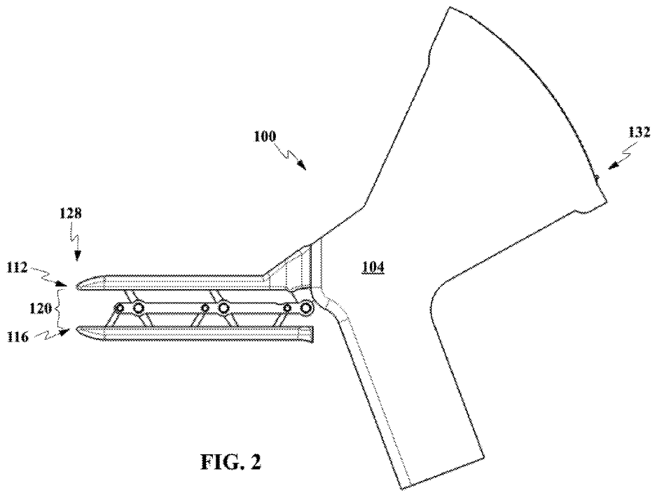

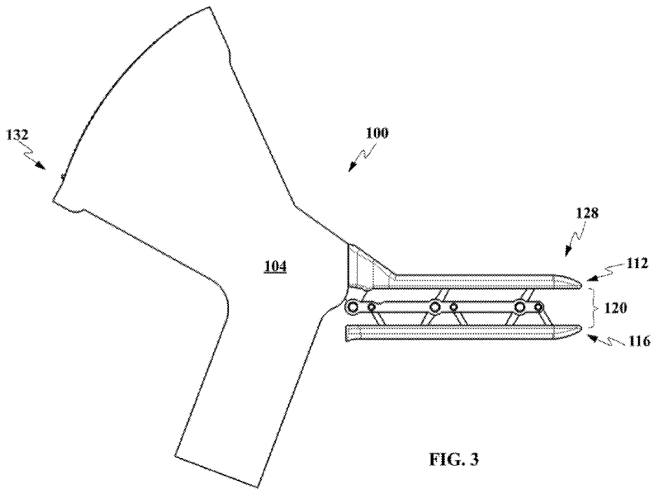

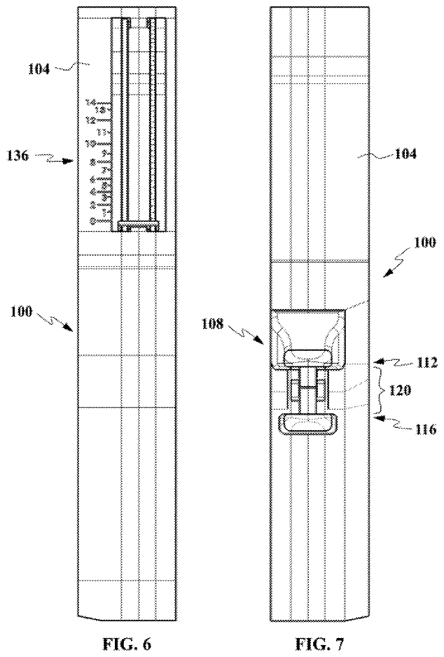

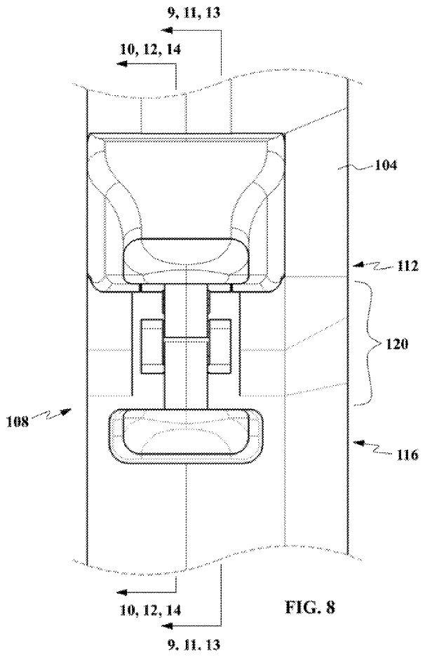

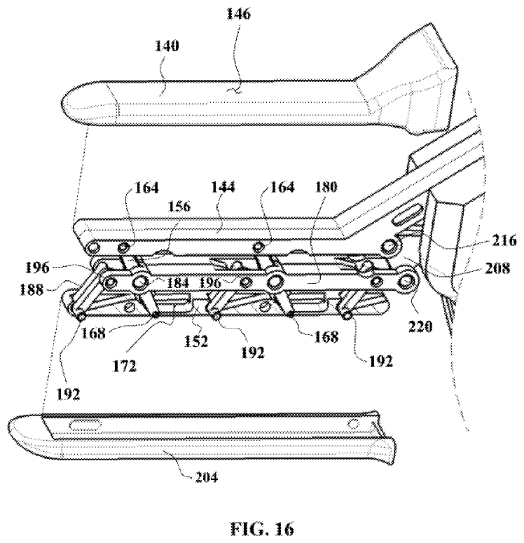

The illustrations of the embodiment in FIGS. 1-20 are produced from a digital 3-D solid model used to 3-D print a device that is exemplary of certain principles according to the invention. While absolute scale may vary between FIGS., elements in each FIG. are internally consistent in scale. One of ordinary skill will appreciate that the illustrations may contain artefacts and certain configurations that reflect the selected manufacturing technique. However, constraints imposed by the 3-D printing process are not to be considered as imposing any limitation on devices according to the invention and manufactured by an alternative process.

An exemplary embodiment of a LeAD device structured according to certain principles of the invention is indicated generally at 100. Device 100 includes a housing 104 coupled to a speculum, generally 108. Speculum 108 includes a first bill, generally 112, and a second bill, generally 116. A guide mechanism, generally 120, operably connects the first and second bills.

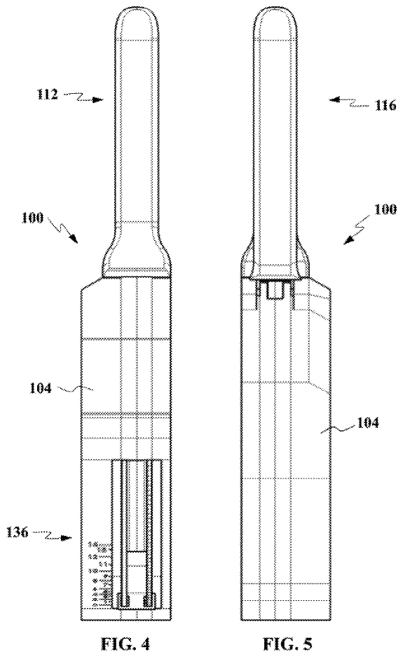

Housing 104 typically includes some sort of a handle, generally 124, to facilitate hand-held operation of the device 100 to measure contraction force generated by pelvic floor muscles in a human. With reference to housing 104, the distal end 128 and proximal end 132 of elements of a device 100 are generally defined relative to proximity to an operator of the device 100. A scale, generally 136 (FIG. 4) may conveniently be carried on a proximal portion of the housing 104.

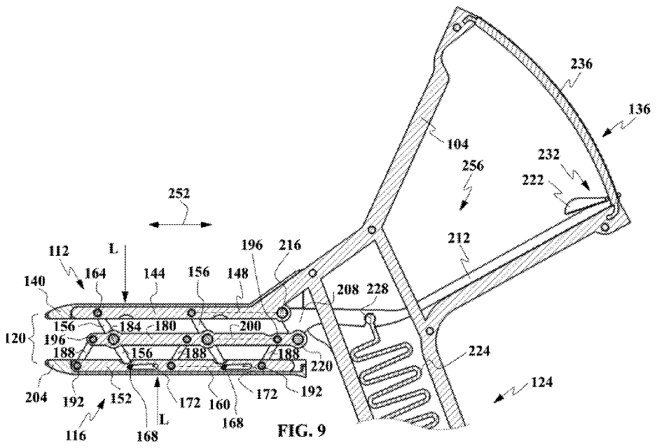

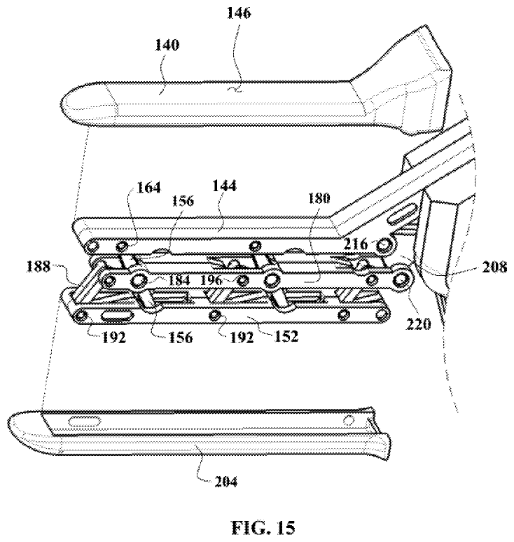

With reference now to FIG. 9, illustrated first bill 112 includes a bill cover 140 carried on first beam 144. The bill cover 140 is configured to present a comfortable surface 146 (see e.g., FIG. 15) for contact with a patient. As illustrated, corners are desirably rounded and the patient contact surface 146 is smooth.

First beam 144 projects as a cantilevered extension from housing 104. As a practical matter, beam 144 may be regarded as a substantially rigid element that is affixed to a rigid housing. That is, a preferred guide mechanism 120 operates, in part, as a support to reduce deflection in beam 144 that might be caused by a transverse load L being applied at various axial positions along a length axis 148 of beam 144. A workable guide mechanism 120 can reduce sensitivity of transverse displacement of the first beam 144 with respect to a second beam 152 due to a position of an applied load L along a length axis of the respective beams.

First beam 144 is connected to a second beam 152 by way of a plurality of primary link elements 156. Similar to beam 140, a preferred guide structure 120 operates, in part, as a distributed support to reduce deflection in beam 152 that might be caused by a transverse load L being applied at various axial positions along a length axis 160 of beam 152.

First ends of primary links 156 are rotatably connected to the first beam 144 by respective axles 164. Opposite ends of primary links 156 are slidingly coupled to beam 152 by way of pins 168 disposed in elongate sockets 172. Sockets 172 are conveniently formed in second beam 152, and are typically configured and arranged to permit pins 168 to reciprocate in parallel with the length axis 160 of second beam 152. Consequently, length axis 148 of the first beam and length axis 160 of the second beam are urged to remain in parallel as the first and second beams deflect toward and away from each other.

Primary links 156 may sometimes be characterized as dual links because they can function as a connection between the first beam 144 and second beam 152, as well as between the first beam 144 and a third beam 180. As illustrated, middles of primary links 156 are rotatably connected to the third beam 180 by respective axles 184. Consequently, the third beam 180 is coupled to the second beam 152 to urge the second and third beams to remain in parallel as the first and second beams deflect toward and away from each other. Primary links 156 also provide a restoring force effective to urge second bill 116 away from first bill 112 when an applied load is removed from the speculum 108.

A partially redundant coupling arrangement between the second beam 152 and the third beam 180 is provided by a plurality of secondary link elements 188. A first end of respective link elements 188 is rotatably connected to second beam 152 by way of axles 192. The opposite end of respective link elements 188 is rotatably connected to third beam 180 by way of axles 196.

The plurality of link elements 188 desirably have the same working length. As a consequence, length axis 160 of second beam 152 is urged to remain parallel to length axis 200 of third beam 180 as the second and third beams deflect transversely toward and away from each other. Second beam 152 carries a bill cover 204 having a similar configuration and functionality to first bill cover 140. Bill covers 140, 204 are arranged so that their patient-contacting surfaces are parallel to each other, and are generally parallel to length axes of their respective carrying beams.

Note that a length axis of the speculum 108 is generally parallel to length axes of the first, second, and third beams. Therefore, in harmony with disclosure of the preceding several paragraphs, an exemplary guide mechanism 120 is configured to urge the first bill 112 and second bill 116 to remain in parallel, irrespective of point of load application along a length axis of the speculum 108.

Still with principle reference to FIG. 9, a link end 208 of pointer arm 212 is configured to operate as a link element to couple first beam 144 and third beam 180. A first end of link end 208 is rotatably connected to first beam 144 by way of axle 216. Axle 216 may sometimes be made reference to as a first arm pivot axle, and is sleeved for rotation about a first arm pivot point that is generally fixed with respect to the housing 104. An opposite second end of link end 208 is rotatably connected to third beam 180 by way of axle 220. Axle 220 may sometimes be made reference to as a driven axle. Driven axle 220 is displaced by a load that is applied to the speculum 108. When a transverse load L is applied to the speculum 108, beam 180 is urged to displace in a proximal direction. Consequently, pointer arm 212 is urged to rotate about axle 216 and carry reading indicator 222 toward a position correlating to the applied load L.

The operable link element formed at link end 208 may sometimes be made reference to as a drive link. As illustrated, drive link 208 has an effective drive link length spanning between a centerline of axle 216 and a centerline of axle 220. A spacing and orientation in space between centers of axle 216 and axle 220 is identical to that between axle 164 and axle 184. Consequently, link end 208 functions as an equivalent link element to a first end of a primary link 156.

A spring 224 is coupled by retaining structure, such as exemplary stopper 228, at a first spring end to pointer arm 212. A second spring end is generally fixed in space with respect to the housing 104. A workable spring 224 is configured and arranged in harmony with pointer arm 212 to bias the first beam 144 and second beam 152 apart. Desirably, spring 224 is also disposed in harmony with the pointer arm 212 to permit quantifiable displacement of an actuator end, generally 232, of the pointer arm 212 responsive to a load being applied to the speculum 108. Illustrated spring 224 is a tension spring. However, it is within contemplation that other spring types, including compression and torsion springs, could be alternatively used with suitable mounting configurations.

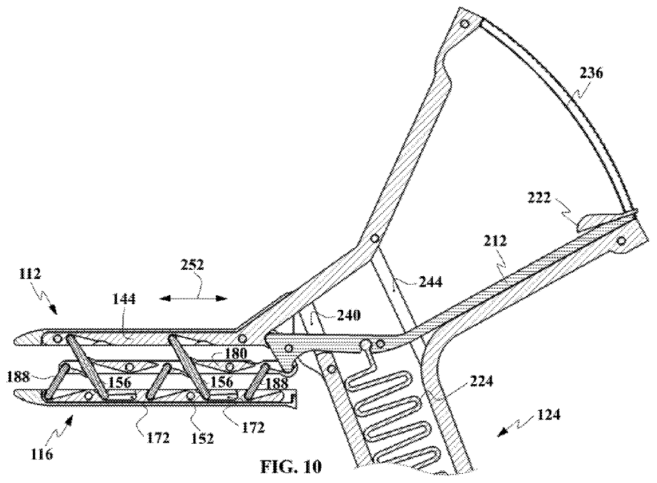

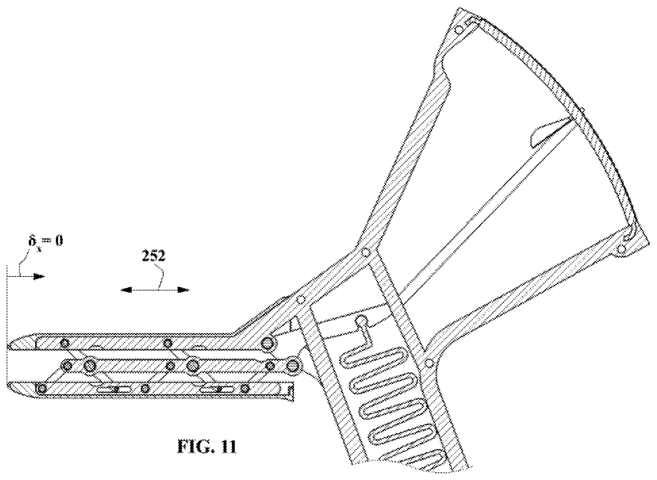

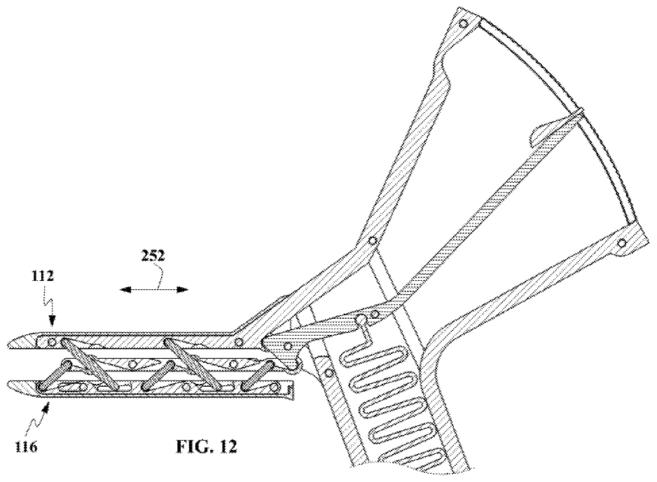

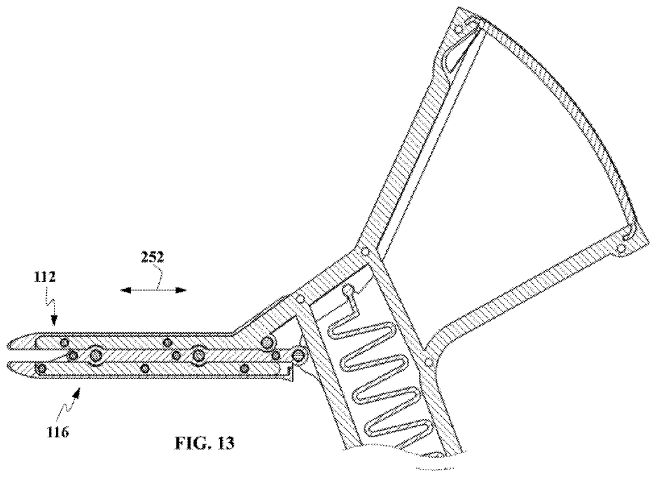

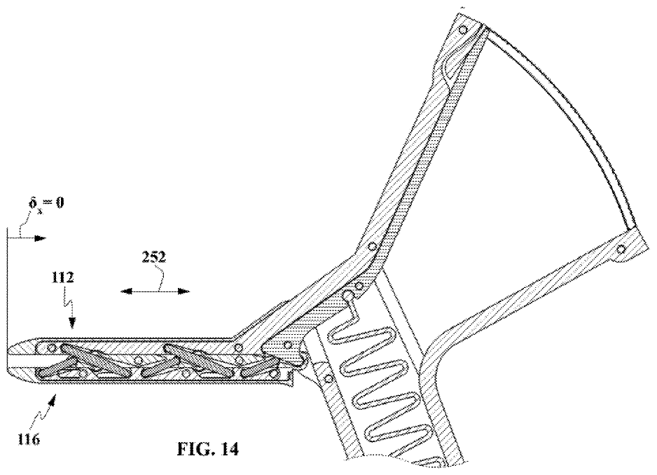

FIGS. 9-10 illustrate embodiment 100 at an unloaded, or zero configuration. It may be observed that first bill 112 is disposed in parallel with second bill 116. FIGS. 11-12 illustrate device 100 at an intermediately displaced configuration. FIGS. 13-14 illustrate device 100 at a fully displaced configuration. It may be perceived that the various linkage elements of guide mechanism 120 urge first bill 112 and second bill 116 to remain parallel to each other throughout an operable range of motion. Further, it can be observed that the first bill 112 and second bill 116 are coupled together by guide mechanism 120 to resist relative axial displacement δₓ between the first bill and the second bill in a bill length direction 252 during actuation of the speculum 108 (e.g., compare absence of relative axial motion δₓ between first and second bills between FIGS. 9, 11, and 14).

With reference now to FIGS. 9-10 and 15-16, it may be appreciated that an exemplary guide mechanism 120 includes an assembly of three multi-bar mechanisms. For purpose of evaluating link action, the substantially rigid housing 104 may be considered as ground. Elements that are operationally rigid and immovably affixed with respect to the housing may also be considered as equivalent to ground.

A first multi-bar mechanism includes a first bar defined by a length of first beam 144 spanning between an axle 164 and an adjacent axle 164. This portion may be considered as effectively ground. A second bar of the first multi-bar mechanism is provided by a first primary link 156 spanning between axle 164 and axle 168 at opposite link ends. A third bar of the first multi-bar mechanism is provided by a second primary link 156 spanning between axle 164 and axle 168 at opposite ends of the second primary link 156. Note, the first and second primary links 156 are constrained to be parallel. A fourth bar of the first multi-bar mechanism is provided by a length of second beam 152 spanning between the adjacent axles 168. In part, this first multi-bar mechanism provides a restoring force to urge second beam 152 away from first beam 144.

A second multi-bar mechanism includes a first bar defined by a length of first beam 144 spanning between an axle 164 and an adjacent axle 164. This portion is shared between the first and second multi-bar mechanisms. A second bar of the second multi-bar mechanism is provided by a portion of a first primary link 156 spanning between an axle 164 and axle 184 at the middle of the first primary link 156. A third bar of the second multi-bar mechanism is provided by a portion of a second primary link 156 spanning between axle 164 and axle 184 at the middle of the second primary link 156. A fourth bar of the second multi-bar mechanism is provided by a length of third beam 180 spanning between the adjacent axles 184. A redundant link 208 between axle 216 and axle 220 may also be considered as a portion of the second multi-bar mechanism.

A third multi-bar mechanism in embodiment 100 includes a first bar defined by a length of third beam 180 between adjacent axles 196. A second bar of the third multi-bar mechanism is defined by a first link element 188 between an axle 196 and axle 192 disposed at respective ends of the link 188. A third bar of the third multi-bar mechanism is defined by a second link element 188 between an axle 196 and axle 192 disposed at respective ends of the second link 188. A fourth bar of the third multi-bar mechanism is provided by a length of the second beam 152 spanning between the adjacent axles 192. A third link 188 may also be considered as a redundant member of the third multi-bar mechanism.

Note that the second and third multi-bar mechanisms are desirably configured to resist generation of relative displacement δₓ in a bill axial length direction 252. Therefore, bar lengths and angular orientation are similar and mirror image, respectively, for vertical parallel bars of the respective mechanisms. Consequently, respective mechanisms are each configured to cause substantially identical relative displacement of the third beam 180 in a proximal direction, responsive to actuation of speculum 108.

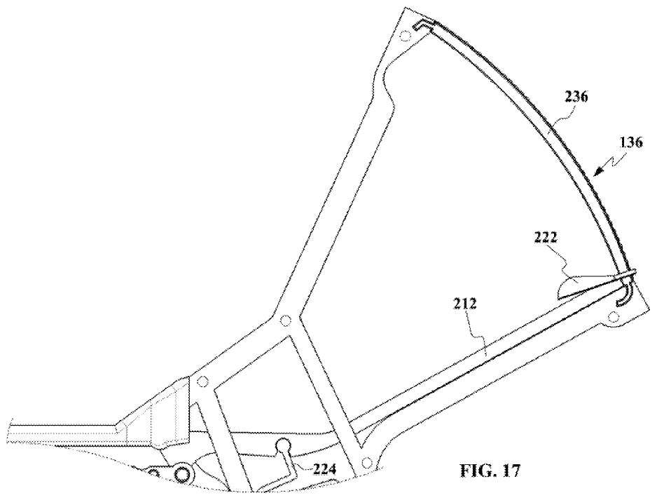

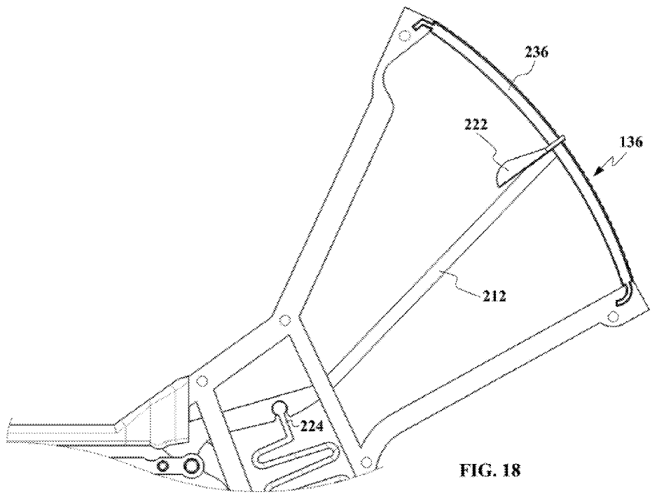

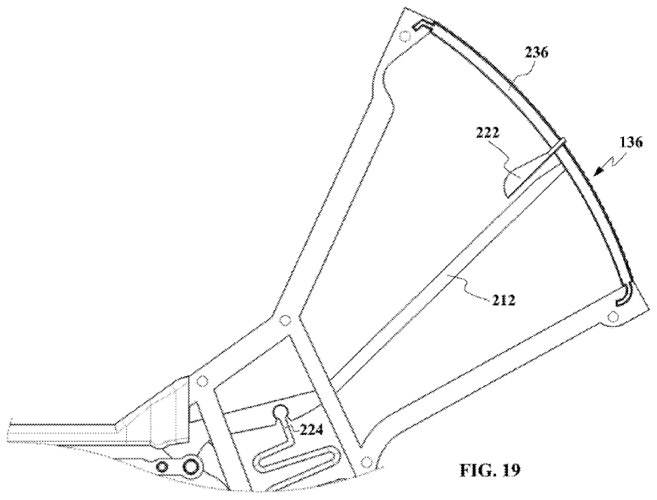

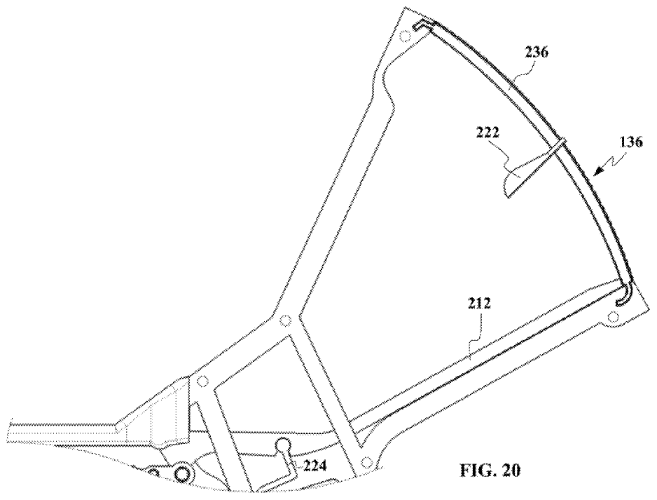

Housing 104 holds a portion of a transducer, generally 256 (FIG. 9). Transducer 256 includes first arm pivot axle 216, driven axle 220, pointer arm 212, and reading indicator 222. As mentioned above, indicator 222 is carried by displacement of actuator end 232 responsive to transverse load L applied on the speculum 108. Pointer arm 212 is disposed for rotation about axle 216 and extends proximally in penetration through first window 240 and second widow 244 (see FIG. 10). Reading indicator 222 is displaced from an initial zero position (FIG. 17) to a variable position (FIG. 18) corresponding to the load magnitude. As the load is reduced, reading indicator 222 rotates and physically couples to arcuate rail 236 under influence of gravity FIG. 19. When the load is completely removed, pointer arm 212 returns to an initial or zero position (FIG. 20). The reading indicator 222 may be physically moved by a clinician to reset the indicator at a zero position for a subsequent reading.

While aspects of the invention have been described in particular with reference to certain illustrated embodiments, such is not intended to limit the scope of the invention. The present invention may be embodied in other specific forms without departing from its spirit or essential characteristics. For one example, one or more element may be extracted from one described or illustrated embodiment and used separately or in combination with one or more element extracted from one or more other described or illustrated embodiment(s), or in combination with other known structure. The described embodiments are to be considered as illustrative and not restrictive. Obvious changes within the capability of one of ordinary skill are encompassed within the present invention. All changes which come within the meaning and range of equivalency of the claims are to be embraced within their scope.

What is claimed is:

1. An apparatus, comprising:
a housing configured to provide a handle for a speculum, the speculum comprising a first bill and a second bill;
the first bill being affixed as a cantilevered beam extension from the housing;
the second bill being coupled to the first bill and constrained by a guide mechanism to permit reciprocating transverse displacement there-between while urging a length axis of the second bill to remain in parallel with a corresponding length axis of the first bill; and
a transducer associated with the speculum to convert a transverse displacement between the first and second bills to a visibly perceptible signal for observation by an operator of the apparatus, the transducer comprising a pointer arm coupled at a first end to a first arm pivot point disposed on a ground element of the cantilever anchor of the first bill or on the first bill, the pointer arm being rotatable about the first arm pivot point responsive to relative transverse displacement between the first and second bills during actuation of the speculum.

2. The apparatus according to claim 1, wherein:
the pointer arm comprises an extended first arm length disposed between the first arm pivot point and an actuator end;
a base end of the pointer arm is configured to provide a drive link comprising a drive link length disposed between the first arm pivot point and a centerline of a driven axle; and
amplified deflection of the actuator end, corresponding to relative displacement between the first and second bills, is a function comprising ratio of first arm length to drive link length.

3. The apparatus according to claim 2, further comprising:
a spring affixed at one spring end to the pointer arm at an intermediate location between the first arm pivot point and the actuator end, and affixed at an opposite spring end to structure corresponding to ground.

4. The apparatus according to claim 3, wherein:
the spring is a tension spring.

5. The apparatus according to claim 3, wherein:
the spring is disposed inside a handle portion of the housing.

6. The apparatus according to claim 2, further comprising:
a scale disposed on a proximal end of the housing to visually indicate a quantified number corresponding to a force applied for actuation of the speculum.

7. The apparatus according to claim 6, wherein:
the scale comprises an arcuate rail and a reading indicator carried for reciprocation on the rail, the actuator end of the pointer arm being operably associated with the reading indicator to displace the indicator along the rail responsive to actuation of the speculum.

8. The apparatus according to claim 7, wherein:
the reading indicator is configured in harmony with the rail and pointer arm to temporarily maintain a displaced position of the reading indicator subsequent to actuation of the speculum by an applied load, and to permit an operator of the apparatus to return the reading indicator to an initial relative zero position subsequent to removal of the load.

9. The apparatus according to claim 2, wherein:
the driven axle is rotatably affixed to a middle beam disposed generally in parallel with and between the first bill and the second bill;
the guide mechanism comprises:
a first multi-bar mechanism arranged to couple the first bill to the middle beam such that length axes of the first bill and middle beam remain in parallel during actuation of the speculum; and
a second multi-bar mechanism arranged to couple the second bill to the middle beam such that length axes of the second bill and middle beam remain in parallel during actuation of the speculum, the first and second multi-bar mechanisms being structured in harmony to maintain the middle beam at a midpoint between the first and second bills; and
the guide mechanism is configured such that the driven axle is operably displaced responsive to a transverse displacement between the first and second bills.

10. The apparatus according to claim 1, wherein:
the guide mechanism is configured to resist relative axial displacement between the first bill and the second bill in a bill length direction during actuation of the speculum.

11. An apparatus, comprising:
a housing configured to provide a handle for handheld use of a speculum;
a speculum projecting from the handle, the speculum comprising a first beam, a second beam, and a third beam;
the first beam being affixed at a proximal end as a cantilevered extension from the housing;
the second beam being coupled to the first beam by way of a plurality of primary link elements to form a first multi-bar mechanism;
the third beam being disposed between the first and second beams and coupled to the first beam by way of the plurality of primary link elements to form a second multi-bar mechanism; and
a transducer associated with the speculum to convert a transverse displacement between the first and second beams to a visibly perceptible signal for observation by an operator of the apparatus.

12. The apparatus according to claim 11, further comprising:
a plurality of secondary link elements coupled for rotation at respective secondary link ends to the second and third beams to form a third multi-bar mechanism.

13. The apparatus according to claim 12, wherein:
the first multi-bar mechanism is constructed and arranged to maintain the first and second beams in a parallel and spaced-apart relation;
the second multi-bar mechanism is constructed and arranged to maintain the first and third beams in parallel; and
the third multi-bar mechanism is constructed and arranged to maintain the second and third beams in parallel.

14. The apparatus according to claim 11, wherein:
primary link first ends are rotatably coupled to the first beam;
primary link middles are rotatably coupled to the third beam; and
primary link second ends are slidably coupled to the second beam to permit primary link second ends to slide with respect to the second beam and in the length direction of the second beam.

15. The apparatus according to claim 12, wherein:
link elements variously disposed between first, second, and third beams inter-cooperate to resist axial displacement of the first beam with respect to the second beam in a length direction of the first beam.

16. The apparatus according to claim 12, wherein:
link elements variously disposed between first, second, and third beams inter-cooperate to cause axial displacement of the third beam with respect to the first and second beams and in a length direction of the third beam.

17. The apparatus according to claim 11, wherein:
axial displacement of the third beam is directed proximally responsive to application of a load to compress the speculum.

18. The apparatus according to claim 11, wherein:
the transducer is carried by the housing and disposed for actuation by the third beam.

19. The apparatus according to claim 11, wherein:
the transducer comprises a pointer arm coupled at a first end to a first arm pivot axle, which defines a pointer rotation axis that is stationary with respect to the housing, the pointer arm being rotatable about the pointer rotation axis responsive to relative transverse displacement between the first and second bills during actuation of the speculum.

20. The apparatus according to claim 11, further comprising:
a spring configured and arranged to bias the first and second beams apart, and coupled to the pointer arm to permit quantifiable displacement of an actuator end of the pointer arm responsive to a load applied to the speculum.
